(19) United States
(12) Patent Application Publication
(10) Pub. No.: US 2026/0232324 A1
(43) Pub. Date: Aug. 13, 2026

(54) STENT WITH ANEURYSM TREATMENT DEVICE

(71) Applicant: MicroVention, Inc., Aliso Viejo, CA (US)

(72) Inventor: Belete Degaga, Aliso Viejo, CA (US)

(73) Assignee: MicroVention, Inc., Aliso Viejo, CA (US)

(21) Appl. No.: 19/161,204

(22) PCT Filed: Mar. 1, 2024
(86) PCT No.: PCT/US2024/018185

§ 371 Date: Aug. 29, 2025

Related U.S. Application Data
(60) Provisional application No. 63/487,826, filed on Mar. 1, 2023.

(51) Int. Cl. A61B 17/12; A61B 17/00; A61B 90/00

(52) U.S. Cl. CPC A61B 17/12118; A61B 17/12145; A61B 90/39; A61B 2017/00477; A61B 2090/3966

(57) ABSTRACT

The present invention discloses an aneurysm treatment device comprising a stent portion and an aneurysm filling portion expanding from an outer side of the stent portion. The device provides a novel approach to treating aneurysms by combining the structural support of a stent with an expandable filling portion to effectively address the needs of patients with aneurysms. The unique design of the device allows for precise placement and expansion within the aneurysm, providing stability and support while promoting healing and recovery. The device offers a versatile and minimally invasive solution for the treatment of aneurysms, improving patient outcomes and reducing the risks associated with traditional treatment methods.

### RELATED APPLICATIONS

This application claims benefit of and priority to U.S. Provisional Application Ser. No. 63/487,826 filed Mar. 1, 2023 entitled Stent With Aneurysm Treatment Device, which is hereby incorporated herein by reference in its entirety.

### BACKGROUND

An aneurysm is typically considered an abnormal bulge or ballooning of a wall of a blood vessel. Aneurysms can rupture or bust, causing internal bleeding and often lead to death of the patient. Hence, it is important to identify and properly treat aneurysms.

Various aneurysm filling devices have been developed to occlude or block off the abnormal dilation within the blood vessel. These filling devices are typically deployed within the aneurysm sac to promote thrombosis and ultimately seal off the aneurysm from blood flow. Some filling devices include microcoils, which may be elongated wire coils, and intrasaccular devices, which may be expandable mesh structures. However, challenges remain in optimizing the design and deployment of such devices to ensure proper positioning, stability, and long-term effectiveness in treating a wide range of aneurysm types and sizes.

### SUMMARY

In some aspects, the techniques described herein relate to an aneurysm treatment device, including: a stent portion; and, a first aneurysm filling portion connected to the stent portion and located on an outside of the stent portion.

In some aspects, the techniques described herein relate to an aneurysm treatment device, wherein the first aneurysm filling portion includes one or more wires forming a three-dimensional spiral coil shape when in an expanded configuration.

In some aspects, the techniques described herein relate to an aneurysm treatment device, wherein the three dimensional spiral coil includes loops that progressively decrease in diameter away from the stent portion or that progressively increase in diameter away from the stent portion when in the expanded configuration.

In some aspects, the techniques described herein relate to an aneurysm treatment device, further including a rounded top portion connected to an end region of the three dimensional spiral coil shape.

In some aspects, the techniques described herein relate to an aneurysm treatment device, further including a rounded top portion connected to an end region of the first aneurysm filling portion; the rounded top portion including a metal, a polymer, or a hydrogel.

In some aspects, the techniques described herein relate to an aneurysm treatment device, wherein the first aneurysm filling portion includes a plurality of braided wires having a three-dimensional shape.

In some aspects, the techniques described herein relate to an aneurysm treatment device, wherein the three-dimensional shape includes a cylindrical shape, a rounded cylindrical shape, a spheroid shape, an oblate spheroid shape, a mound shape, a rounded dome shape, or a rounded pyramid shape.

In some aspects, the techniques described herein relate to an aneurysm treatment device, further including a second aneurysm treatment portion; wherein the second aneurysm treatment portion is positioned on the outside of the stent portion radially opposite the first aneurysm treatment portion.

In some aspects, the techniques described herein relate to an aneurysm treatment device, further including a second aneurysm treatment portion; wherein the second aneurysm treatment portion is positioned on the outside of the stent portion and longitudinally adjacent the first aneurysm treatment portion.

In some aspects, the techniques described herein relate to an aneurysm treatment device, further including a second aneurysm treatment portion, a third aneurysm treatment portion, and a fourth aneurysm treatment portion positioned on the outside of the stent portion; wherein the first aneurysm treatment portion, the second aneurysm treatment portion, the third aneurysm treatment portion, and the fourth aneurysm treatment portion are positioned along a circular pattern perpendicular to a longitudinal axis of the stent portion.

In some aspects, the techniques described herein relate to an aneurysm treatment device, further including a plurality of radiopaque markers aligned at locations forming a pattern around a circumference of the stent portion, and near or between a distal edge of the aneurysm filling portion and/or near or between a proximal edge of the first aneurysm treatment portion.

In some aspects, the techniques described herein relate to an aneurysm treatment device, further including a plurality of radiopaque markers aligned at locations forming a pattern circumferentially around the first aneurysm filling portion.

In some aspects, the techniques described herein relate to an aneurysm treatment device, wherein the first aneurysm filling portion includes radiopaque DFT wires.

In some aspects, the techniques described herein relate to an aneurysm treatment device, wherein the first aneurysm filling portion is attached to the stent portion by welding, adhesives, attachment coils, or by interweaving through each other.

In some aspects, the techniques described herein relate to an aneurysm treatment device, wherein the first aneurysm filling portion has a compressed configuration and a radially expanded configuration that expands radially away from the stent portion.

In some aspects, the techniques described herein relate to an aneurysm treatment device, including: a stent portion including one or more stent wires woven into a tubular shape; and, a first aneurysm filling portion connected to the stent portion and located on an outside of the stent portion; wherein the first aneurysm filling portion has a compressed configuration and an expanded configuration forming a three-dimensional shape; and wherein the three-dimensional shape includes a cylindrical shape, a rounded cylindrical shape, a spheroid shape, an oblate spheroid shape, a mound shape, a rounded dome shape, or a rounded pyramid shape.

In some aspects, the techniques described herein relate to an aneurysm treatment device, wherein the first aneurysm filling portion is one of a plurality of aneurysm filling portions located on an outside of the stent portion.

In some aspects, the techniques described herein relate to an aneurysm treatment device, wherein the plurality of aneurysm filling portions are arranged in one or more longitudinal patterns, one or more circumferential patterns, or both.

In some aspects, the techniques described herein relate to an aneurysm treatment device, further including a plurality of radiopaque markers positioned on the stent portion; the plurality of radiopaque markers being positioned near a perimeter of the first aneurysm filling portion.

In some aspects, the techniques described herein relate to an aneurysm treatment device, including: a stent portion; and, a means for filling an aneurysm that is connected to the stent portion and located on an outside of the stent portion.

In some aspects, the techniques described herein relate to a method of delivering an aneurysm treatment device including: positioning an aneurysm treatment device near a mouth of an aneurysm; moving the aneurysm treatment device out of a delivery catheter so that a stent portion of the aneurysm treatment device radially expands and anchors within a blood vessel; and, expanding an aneurysm filling portion of the aneurysm treatment device into the aneurysm.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 illustrates delivery of a flow diverter stent and one or more microcoils.

FIG. 2 illustrates a side view of an aneurysm treatment device according to one example.

FIG. 3 illustrates the aneurysm treatment device of FIG. 2 in a second rotational orientation according to one example.

FIG. 4 illustrates the aneurysm treatment device of FIG. 2 in a crimped or a radially compressed configuration within a delivery catheter according to one example.

FIG. 5 illustrates the aneurysm treatment device of FIG. 2 within a blood vessel and aneurysm, according to one example.

FIG. 6 illustrates a side view of another aneurysm treatment device according to one example.

FIG. 7 illustrates the aneurysm treatment device of FIG. 6 in a second rotational orientation according to one example.

FIG. 8 illustrates the aneurysm treatment device of FIG. 6 in a crimped or a radially compressed configuration within a delivery catheter according to one example.

FIG. 9 illustrates a side view of the aneurysm treatment device of FIG. 6 deployed within a blood vessel and aneurysm of a patient according to one example.

FIG. 10 illustrates a side view of another aneurysm treatment according to one example.

FIG. 11 illustrates the aneurysm treatment device of FIG. 10 deployed in a fusiform aneurysm according to one example.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Presently, there are several different types of treatments for aneurysms that may generally include filling the aneurysm and/or blocking off blood flow to the aneurysm from the main vessel.

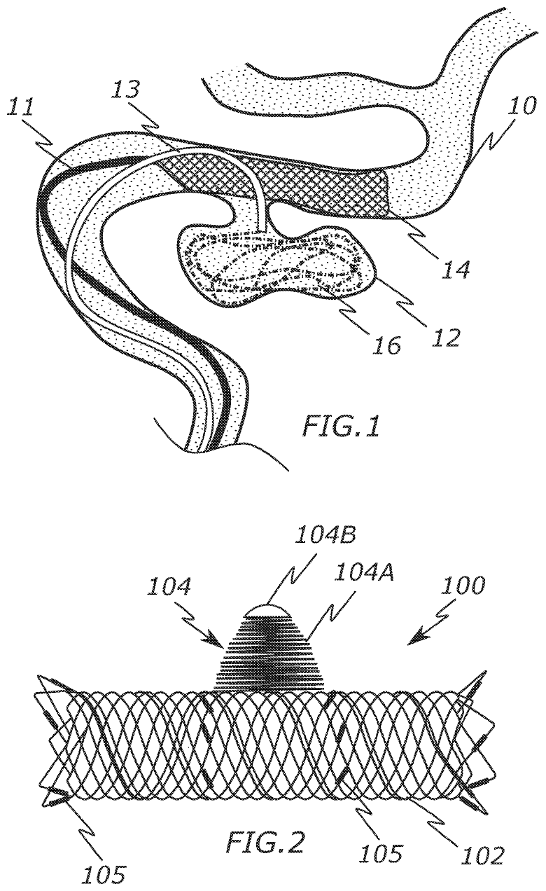

FIG. 1 illustrates delivery of a flow diverter stent 14 and one or more microcoils 16. In particular, FIG. 1 illustrates one common treatment for an aneurysm 12 within a blood vessel 10. A first catheter 11 may deliver a flow diverter stent 14 across the mouth of the aneurysm 12. A second catheter 13 may then deliver material, such as one or more microcoils 16, to at least partially fill the interior of the aneurysm 12. The flow diverter stent 14 may help maintain the material (e.g., microcoils 16) within the aneurysm 12 while also limiting or preventing blood from entering the aneurysm 12.

Since multiple catheters are typically used and since multiple devices are typically deployed, such a procedure may require a significant amount of time and skill to perform. Additionally, such a procedure may encounter certain difficulties. For example, when advancing microcoils 16 out of the second catheter 13, the distal end of the second catheter 13 may be moved or kicked out of the aneurysm 12 from the force of the exiting microcoils.

The present disclosure is generally directed to an aneurysm treatment device that combines a flow diverting stent with an aneurysm filling portion as a single device that can be deployed at the same time and via a single catheter. Hence, aneurysm treatments may require less equipment (e.g., less catheters) and less time to perform.

The aneurysm treatment device may include a stent portion that has a tubular body with a lumen extending through it. The stent portion can be a generally cylindrical shape or may branch into a Y shape, depending on the configuration of the vessels near the aneurysm. The stent portion may be formed from one or more wires braided together, one or more coiled wires (e.g., one or more helical wires), or may be formed from a plurality of laser-cut struts.

The stent portion may be configured to partially or fully block blood flow through its walls, though not through its main lumen (e.g., a flow diverter stent). Such blockage may be achieved by creating the stent with a relatively low porosity (e.g., via braiding, or an inner or outer layer).

The stent portion may expand from a relatively radially compressed configuration to a relatively radially expanded configuration. This expansion may be caused by the materials of the stent portion that allow it to be self-expanding (e.g., shape-set Nitinol) or may be balloon expandable.

The aneurysm filling portion may be connected to an outside location of the stent portion (e.g., proximal end portion, distal end portion, or middle portion). The stent portion may have an opening at the location of the aneurysm filling portion or may not have an opening at the location of the aneurysm filling portion (i.e., the interior of the two portions are not connected to each other).

The aneurysm filling portion may have a compressed or low profile configuration and an expanded or enlarged configuration. The aneurysm filling portion may be composed of materials that allow it to be self-expanding (e.g., shape set Nitinol) or may be expandable by other components (e.g., balloon expandable). Depending on the structure of the aneurysm filling portion, it may compress against the wall of the stent portion and the interior of the delivery catheter and/or may fold proximally or distally against the wall of the stent portion.

The aneurysm filling portion may have a variety of different structures. For example, the aneurysm filling portion may comprise one or more coiled wires (e.g., straight wires or microcoils) arranged in a spiral, conical, and/or dome shape. In another example, the aneurysm filling portion may comprise a plurality of braided wires that are structured and optionally shape-set to expand to a conical or dome shape. Any of these aneurysm filling portions may optionally have a distal end cap that is solid (e.g., a membrane) or porous (e.g., mesh or a porous membrane).

The aneurysm filling portion may have a variety of different shapes, including a generally spherical shape, cylindrical shape, a spheroid, an oblate spheroid, a mound shape, a pyramid with a rounded top, or similar shapes.

The aneurysm filling portion may be connected to the stent portion via wire coils, loops, clips, hooks, adhesive, welding, or similar techniques. The aneurysm filling portion may also be integrally woven with wires continuing on from the stent portion (i.e., a continuous braided 3D shape) or one of the portions may be woven through the already braided wires of the other portion (i.e., two discretely woven portions).

The aneurysm treatment device may include one or more radiopaque markers that are positioned on the stent portion near the aneurysm filling portion so as to convey the location of the aneurysm filling portion via visualization procedures during a procedure, thereby allowing a physician to align the aneurysm filling portion with a mouth of an aneurysm. The radiopaque markers may align radially around the tubular structure of the stent and/or may encircle the aneurysm filling portion. The radiopaque markers may be wire coils, bands, or similar structures. The radiopaque markers may be one or more wires interwoven with the wires of the stent portion (e.g., radially around the stent body, in a circular pattern around the aneurysm filling portion, or longitudinally along the stent portion for about the length of the aneurysm filling portion. Alternatively, the aneurysm treatment portion may be composed of radiopaque wires or material. Alternatively, the aneurysm treatment portion may comprise one or more radiopaque markers either instead of the radiopaque markers on the stent portion or in addition to any markers on the stent portion. The stent portion may also include radiopaque markers at its proximal and/or distal ends.

In use, the aneurysm treatment device is located within a delivery catheter in its compressed configuration. The distal end of the delivery catheter may be positioned near a mouth of an aneurysm and the aneurysm filling portion may be aligned with the mouth of the aneurysm. The aneurysm treatment device is advanced out of the delivery catheter (or an outer sheath on the delivery catheter is proximally withdrawn), allowing the stent portion to expand within the vessel and the aneurysm filling portion to expand within the aneurysm.

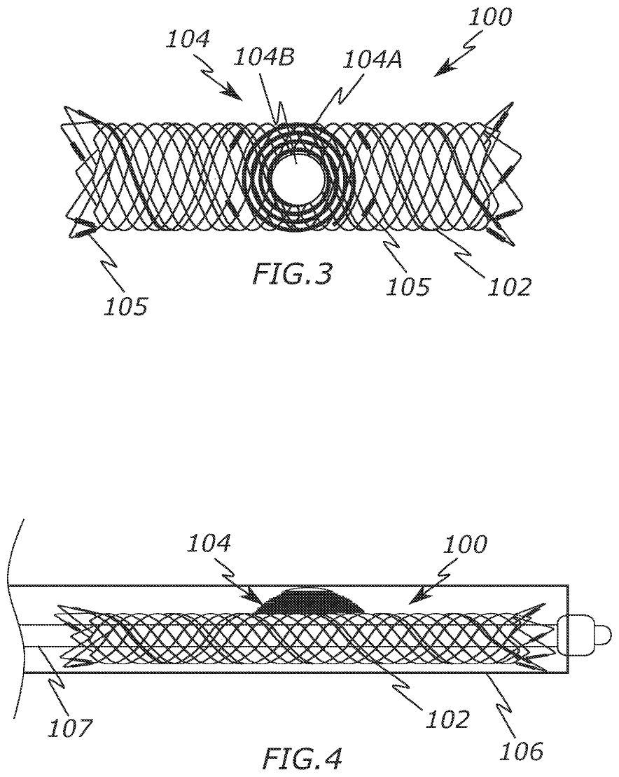

FIGS. 2 and 3 illustrate an aneurysm treatment device 100 according to one example. FIG. 2 illustrates the aneurysm treatment device 100 in a first rotational orientation and FIG. 3 illustrates the aneurysm treatment device 100 in a second rotational orientation that is rotated about 90 degrees relative to FIG. 1. Both figures are discussed concurrently below.

FIGS. 2 and 3 illustrate a specific example of an aneurysm treatment device 100 comprising a tubular stent portion 102 and an aneurysm filling portion 104 connected to and extending from the stent portion 102. As discussed further below, the aneurysm treatment device 100 may be deployed such that the stent portion 102 radially expands within a vessel and the aneurysm filling portion 104 expands within an aneurysm connected to the vessel. In this respect, only a single device may need to be deployed to treat an aneurysm as opposed to a separate stent and separate aneurysm filling material.

In the present example, the stent portion 102 is formed from one or a plurality

of braided wires that form a cylindrical, tubular shape. However, other stent constructions, such as laser-cut stents, are also possible. In one example, the stent portion 102 is configured as a flow diverting stent portion that partially or fully blocks or diverts blood flow through its walls (though not through its lumen). Examples stents and features that may be used as the stent portion 102 or part of the stent portion 102 include U.S. Pat. Nos. 9,439,791 and 10,039,655, which are hereby incorporated herein by reference.

In some examples, the aneurysm filling portion 104 is comprised of one or more coiled wires 104A that have an expanded shape of a dome or mound (or alternatively any of the other shapes described in this disclosure). In the example of FIGS. 2 and 3, a single wire forms loops that progressively decrease in diameter towards a center of the aneurysm filling portion 104. Portions of the widest diameter loops may be attached to the stent portion 102 in any manner discussed in this disclosure, such as via welding, wire coils, or interweaving with wires of the stent portion 102. The one or more coiled wires 104A may have a memory shape or configuration in which it expands to its expanded shape such that the smallest loops are positioned furthest from the stent portion 102 or progressively decrease in size away from the stent portion 102 (e.g., the dome, mound, or three-dimensional spiral coil shape in an expanded configuration in FIGS. 2 and 3).

Alternatively, the one or more coiled wires 104A may have loops that generally maintain the same diameter and form a cylindrical expanded shape. In another alternative example, the one or more coiled wires 104A may have loops that progressively increase in diameter away from the stent portion 102 (e.g., an inverted cone), such that portions of the smaller diameter loops are connected to the stent portion 102.

In some examples, the top of the one or more coiled wires 104A may include a rounded or domed top portion 104B that may be connected to an end region of the one or more wires (i.e., an end region positioned away from the stent portion 102) to prevent them from damaging or rupturing the aneurysm 12. The rounded or domed top portion 104B may be composed of almost any material and shape that may be configured in an atraumatic configuration. For example, the material may be a nonporous material or a porous material such as a braided mesh or porous membrane. The rounded or domed top portion 104B may be composed of a metal (e.g., Nitinol or a radiopaque metal), a polymer (e.g., PET), a hydrogel, or similar materials.

The aneurysm treatment device 100 may also include radiopaque markers 105 to help define the location of the aneurysm filling portion 104 during a procedure (e.g., using fluoroscopy). The radiopaque markers 105 may form several different patterns. As seen in FIG. 2, the radiopaque markers 105 may be aligned at locations forming a pattern around the circumference or perimeter of the stent portion 102 (e.g., forming a circular pattern that is generally perpendicular to a longitudinal axis of the stent portion 102). Such a circular pattern may be located proximally and/or distally of the aneurysm filling portion 104 (e.g., near or between a distal and/or proximal edge of the aneurysm filling portion 104), as well as anywhere in between. As seen in FIG. 3, radiopaque markers 105 may also or alternatively be aligned at locations forming a pattern circumferentially around the aneurysm filling portion 104. The radiopaque markers 105 may also be located at one or more ends of the stent portion 102.

FIG. 4 illustrates the aneurysm treatment device 100 in a crimped or a radially compressed configuration within a delivery catheter 106. In some examples, the aneurysm treatment device 100 may be compressed over an inner pusher 107 that may be used to advance the aneurysm treatment device 100 out of a distal end of the delivery catheter 106 or alternatively the delivery catheter 106 may be proximally withdrawn relative to the inner pusher 107. In some examples, the aneurysm filling portion 104 may be compressed against the wall of the stent portion 102 when in the delivery catheter 106. In the present example of FIG. 4, the aneurysm filling portion 104 is compressed generally perpendicularly relatively to a longitudinal axis of the stent portion 102, however, the aneurysm filling portion 104 may also or alternatively be compressed and/or arranged longitudinally along the stent portion 102.

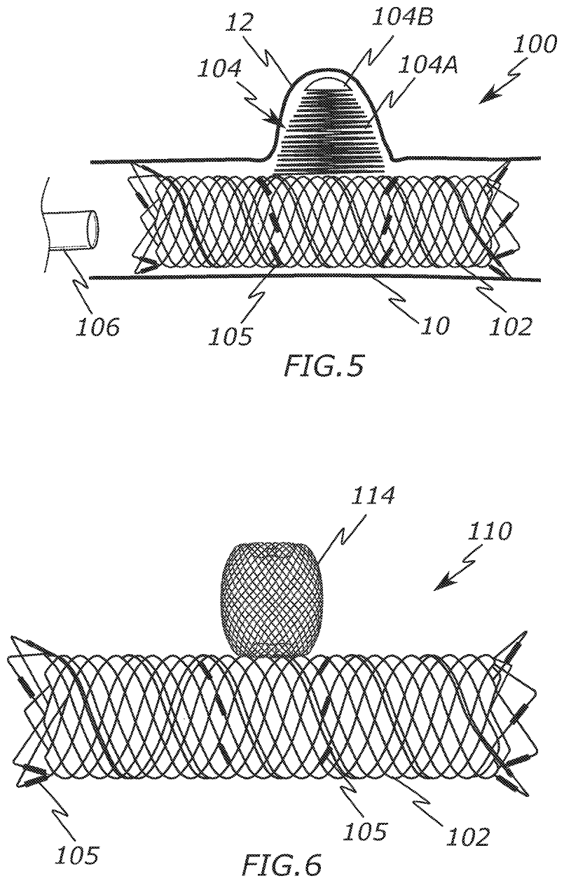

FIG. 5 illustrates the aneurysm treatment device 100 from FIG. 4 after it has been delivered within a blood vessel 10 and aneurysm 12, according to one example. Prior to the placement of the aneurysm treatment device 100, the delivery catheter 106 may first be aligned to a desired position within the blood vessel 10 and relative to the aneurysm 12. Depending on whether the delivery catheter 106 will be proximally retracted relative to the inner pusher 107 or whether the inner pusher 107 will be distally advanced out of the distal end of the delivery catheter 106, the distal end or region of the delivery catheter 106 may be positioned either underneath the aneurysm 12 or adjacent to the aneurysm 12. The positioning of the aneurysm treatment device 100 and particularly the aneurysm filling portion 104 may be achieved by viewing the one or more radiopaque markers 105 during the procedure to help align the aneurysm filling portion 104 to a desired position (e.g., directly underneath the aneurysm 12). As the aneurysm treatment device 100 is released, the stent portion 102 radially expands against the walls of the blood vessel 10 while the aneurysm filling portion 104 perpendicularly expands into the aneurysm 12.

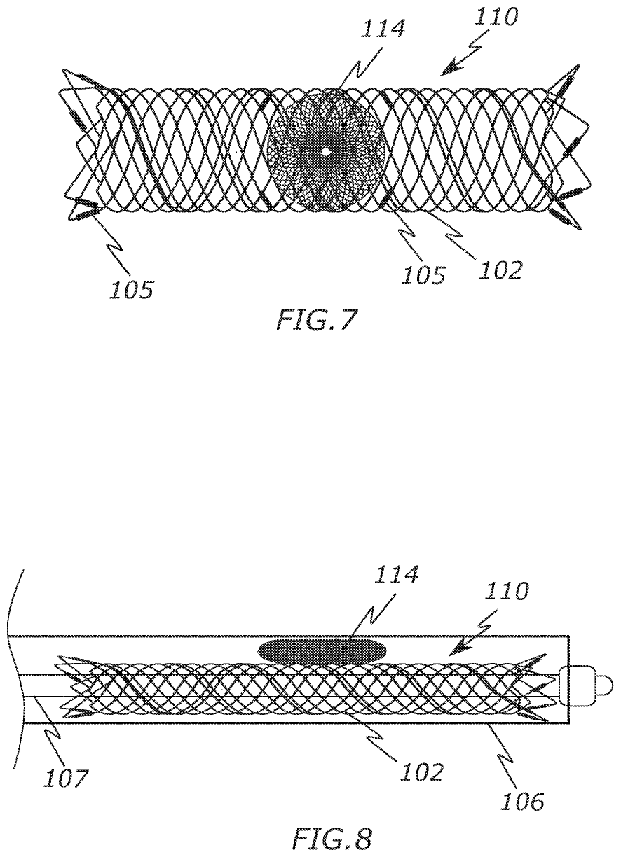

As previously discussed, the aneurysm filling portion may take several different forms. FIGS. 6 and 7 illustrate another example of an aneurysm treatment device similar to the previously described aneurysm treatment device 100 with an aneurysm filling portion 114. FIG. 6 illustrates a side view of the aneurysm treatment device 110 in a first rotational orientation while FIG. 7 illustrates a side view of the aneurysm treatment device 100 in a second rotational orientation that is about 90 degrees relative to the first rotational orientation.

In the present example, the aneurysm treatment device 110 may include a stent portion 102 similar to that previously discussed with regard to the aneurysm treatment device 100 and elsewhere in this disclosure.

In the present example, the aneurysm filling portion 114 may comprise a mesh structure having a compressed configuration against the stent portion 102 and an expanded configuration in which it extends perpendicularly away from the stent portion 102. The mesh structure may comprise one or more wires braided or woven together to form a three-dimensional shape in its expanded configuration. In one example, the one or more wires may be a shape memory material such as Nitinol or a DFT wire comprising both a shape memory material and a radiopaque material. The shape memory materials may allow the aneurysm filling portion 114 to be shape-set to form the desired expanded configuration shape.

In the expanded configuration, the aneurysm filling portion 114 may form a variety of different shapes, such as a spherical shape, a cylindrical shape, a rounded cylindrical shape, a spheroid shape, an oblate spheroid shape, a rounded dome shape, a rounded pyramid shape, similar shapes, or other shapes described in this disclosure. Any of these shapes may be hollow or may include additional material or mesh therewithin.

The top of the aneurysm filling portion 114 may be rounded or otherwise atraumatic in shape, such as seen in FIG. 6. The top of the aneurysm filling portion 114 may be braided such that no free ends of the one or more wires come together at its top, or if any free ends do come together, they take an atraumatic form (e.g., a rounded union point and/or a top depression to accommodate any union point). Additionally or alternatively, the aneurysm filling portion 114 may include a rounded or domed top portion similar to the rounded or domed top portion 104B.

Additional shapes, manufacturing techniques, and other details of the aneurysm filling portion 114 may be found in U.S. Pat. Nos. 9,629,635 and 9,955,976; the contents of which are hereby incorporated herein by reference.

As seen in FIGS. 6 and 7, the aneurysm filling portion 104 may also include radiopaque markers 105 in a similar manner as discussed with regard to aneurysm treatment device 100. These radiopaque markers 105 may help a physician view and therefore align the aneurysm filling portion 114 during a procedure.

The aneurysm filling portion 114 may be attached to the stent portion 102 in any manner described in this disclosure. For example, a plurality of attachment wires or coils may be connected to the wires of both the aneurysm filling portion 114 and the stent portion 102. In another example, the stent portion 102 or the aneurysm filling portion 114 may be braided or woven into the other during manufacture. In another example, the aneurysm filling portion 114 may be welded or attached via adhesive to the stent portion 102.

FIG. 8 illustrates a side view of the aneurysm treatment device 110 of FIG. 6 in a crimpled or in a radially compressed configuration. The aneurysm treatment device 110 is shown crimped or compressed on an inner pusher 107 within a delivery catheter 106. Hence, the delivery catheter 106 may help radially restrain both the stent portion 102 and the aneurysm filling portion 114 until delivery within the patient.

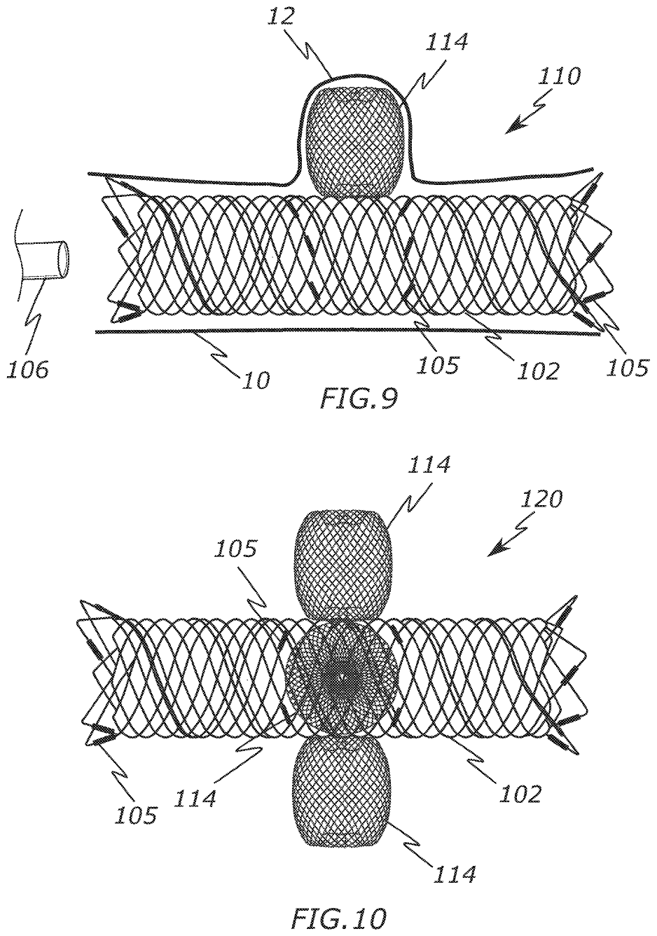

FIG. 9 illustrates a side view of the aneurysm treatment device 110 of FIG. 6 deployed within a blood vessel 10 and aneurysm 12 of a patient. Prior to the placement of the aneurysm treatment device 110, the delivery catheter 106 may first be aligned to a desired position within the blood vessel 10 and relative to the aneurysm 12. Depending on whether the delivery catheter 106 will be proximally retracted relative to the inner pusher 107 or whether the inner pusher 107 will be distally advanced out of the distal end of the delivery catheter 106, the distal end or region of the delivery catheter 106 may be positioned either underneath the aneurysm 12 or adjacent to the aneurysm 12. The positioning of the aneurysm treatment device 110 and particularly the aneurysm filling portion 114 may be achieved by viewing the one or more radiopaque markers 105 during the procedure to help align the aneurysm filling portion 114 to a desired position (e.g., directly underneath the aneurysm 12). As the aneurysm treatment device 110 is released, the stent portion 102 radially expands against the walls of the blood vessel 10 while the aneurysm filling portion 114 perpendicularly expands into the aneurysm 12.

Any of the examples within this disclosure may have either one aneurysm filling portion or a plurality of aneurysm filling portions. For example, two or more aneurysm filling portions may be positioned longitudinally near or longitudinally adjacent to each other along the length of the stent portion 102 (e.g., in a line parallel a longitudinal axis of the stent portion 102). In another example, two or more aneurysm treatment portions may be located at different radial positions of the stent portion 102 (e.g., along a circular pattern that is perpendicular to a longitudinal axis of the stent portion 102). Any combination of these longitudinal positions and radial positions may be combined together to accommodate an aneurysm shape and size. For example, 2, 3, 4, 5, 6, 7, 8 or more aneurysm filling portions may be located in a longitudinal array along a length of the stent portion 102 in a linear pattern. In another example, 2, 3, 4, 5, 6, 7, 8 or more aneurysm filling portions may be located in a circular pattern around the stent portion 102. Any combinations of the longitudinal and circular patterns may be combined and may be symmetrically positioned along the stent portion 102 or non-symmetrically positioned. Other patterns are also possible, such as a plurality of aneurysm filling portions located in a helical pattern along the length of the stent portion 102.

In one specific example, FIG. 10 illustrates a side view of another aneurysm treatment device 120 which is generally similar to the previously described aneurysm treatment device 110 but includes a plurality of aneurysm filling portions 114. Specifically, the aneurysm treatment device 120 includes four aneurysm filling portions 114 positioned circumferentially and symmetrically around the stent portion 102 (e.g., two different aneurysm filling portions 114 positioned directly opposite each other and two different aneurysm filling portions 114 at about 90 degrees to the first pair).

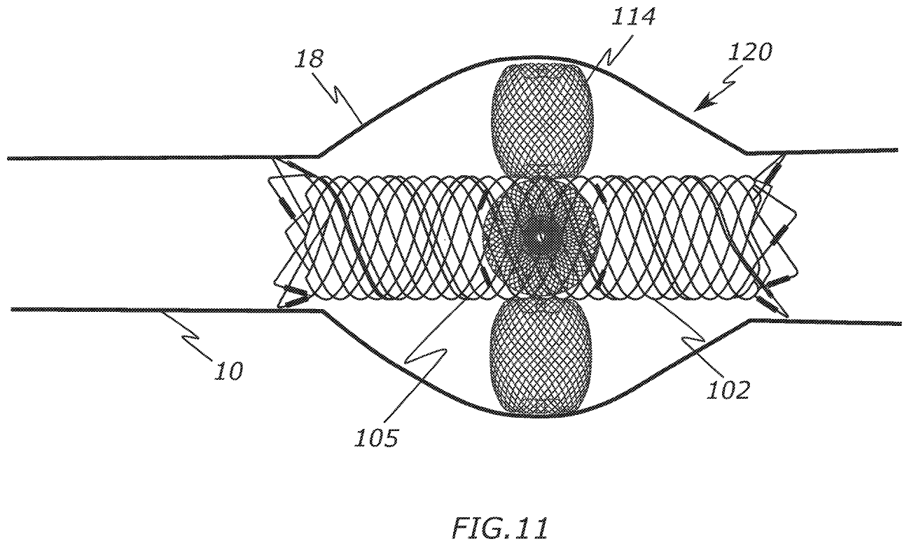

FIG. 11 illustrates the aneurysm treatment device 120 of FIG. 10 deployed in a fusiform aneurysm 18. A fusiform aneurysm 18 typically has a circumferentially expanding shape relative to adjacent portions of a blood vessel, as opposed to other shapes such as the saccular shape of aneurysm 12 illustrated in prior figures. Since fusiform aneurysms 18 have a circumferentially enlarged shape, they can be relatively more difficult to fill and otherwise treat. For example, an elongated stent may be placed across the entire fusiform aneurysm 18 and then the circumferential gap between the stent and the wall of the fusiform aneurysm 18 may be filled with a filling material (e.g., microcoils). Such a procedure may be challenging to perform without undesirably moving the stent and adequately filling all areas around the stent.

The present aneurysm treatment device 120 and variations thereof with a plurality of aneurysm filling portions 114 may increase the ease and time for deployment since the stent portion 102 is deployed simultaneously with the aneurysm filling portions 114. Additionally, the aneurysm filling portions 114 are deployed circumferentially around the stent portion 102 to help fill in and possibly further anchor the 120 within the fusiform aneurysm 18.

The aneurysm treatment device 120 may also be deployed to treat aneurysms of other shapes, such as a saccular shaped aneurysm 12. In some examples, each of the aneurysm filling portions 114 may expand independently of each other. Hence, when a physician deploys the aneurysm treatment device 120, some of the aneurysm filling portions 114 may expand into an aneurysm that they are aligned with while other aneurysm filling portions 114 may be generally maintained in a non-expanded configuration by the wall of the blood vessel. The exact radial orientation of the aneurysm treatment device 120 may require less accuracy such that most rotational orientations will result in at least one (but possibly two or more) aneurysm filling portions 114 expanding into an aneurysm while the others remain compressed. Additionally, some patients may have two or more aneurysms near each other, which some of the multiple aneurysm filling portions may be able to expand into instead of using two or more treatment devices. Again, any aneurysm filling portions 114 that are not aligned with one of the aneurysms may remain in their compressed configurations.

### Claim Bank

Clause 1. An aneurysm treatment device, comprising: a stent portion; and, a first aneurysm filling portion connected to the stent portion and located on an outside of the stent portion.

Clause 2. The aneurysm treatment device of clause 1, wherein the first aneurysm filling portion comprises one or more wires forming a three-dimensional spiral coil shape when in an expanded configuration.

Clause 3. The aneurysm treatment device of clause 2, wherein the three dimensional spiral coil comprises loops that progressively decrease in diameter away from the stent portion or that progressively increase in diameter away from the stent portion when in the expanded configuration.

Clause 4. The aneurysm treatment device of clause 3, further comprising a rounded top portion connected to an end region of the three dimensional spiral coil shape.

Clause 5. The aneurysm treatment device of clause 1, further comprising a rounded top portion connected to an end region of the first aneurysm filling portion; the rounded top portion comprising a metal, a polymer, or a hydrogel.

Clause 6. The aneurysm treatment device of clause 1, wherein the first aneurysm filling portion comprises a plurality of braided wires having a three-dimensional shape.

Clause 7. The aneurysm treatment device of clause 6, wherein the three-dimensional shape comprises a cylindrical shape, a rounded cylindrical shape, a spheroid shape, an oblate spheroid shape, a mound shape, a rounded dome shape, or a rounded pyramid shape.

Clause 8. The aneurysm treatment device of clause 1, further comprising a second aneurysm treatment portion; wherein the second aneurysm treatment portion is positioned on the outside of the stent portion radially opposite the first aneurysm treatment portion.

Clause 9. The aneurysm treatment device of clause 1, further comprising a second aneurysm treatment portion; wherein the second aneurysm treatment portion is positioned on the outside of the stent portion and longitudinally adjacent the first aneurysm treatment portion.

Clause 10. The aneurysm treatment device of clause 1, further comprising a second aneurysm treatment portion, a third aneurysm treatment portion, and a fourth aneurysm treatment portion positioned on the outside of the stent portion; wherein the first aneurysm treatment portion, the second aneurysm treatment portion, the third aneurysm treatment portion, and the fourth aneurysm treatment portion are positioned along a circular pattern perpendicular to a longitudinal axis of the stent portion.

Clause 11. The aneurysm treatment device of clause 1, further comprising a plurality of radiopaque markers aligned at locations forming a pattern around a circumference of the stent portion, and near or between a distal edge of the aneurysm filling portion and/or near or between a proximal edge of the first aneurysm treatment portion.

Clause 12. The aneurysm treatment device of clause 1, further comprising a plurality of radiopaque markers aligned at locations forming a pattern circumferentially around the first aneurysm filling portion.

Clause 13. The aneurysm treatment device of clause 1, wherein the first aneurysm filling portion comprises radiopaque DFT wires.

Clause 14. The aneurysm treatment device of clause 1, wherein the first aneurysm filling portion is attached to the stent portion by welding, adhesives, attachment coils, or by interweaving through each other.

Clause 15. The aneurysm treatment device of clause 1, wherein the first aneurysm filling portion has a compressed configuration and a radially expanded configuration that expands radially away from the stent portion.

Clause 16. An aneurysm treatment device, comprising: a stent portion comprising one or more stent wires woven into a tubular shape; and, a first aneurysm filling portion connected to the stent portion and located on an outside of the stent portion; wherein the first aneurysm filling portion has a compressed configuration and an expanded configuration forming a three-dimensional shape; and wherein the three-dimensional shape comprises a cylindrical shape, a rounded cylindrical shape, a spheroid shape, an oblate spheroid shape, a mound shape, a rounded dome shape, or a rounded pyramid shape.

Clause 17. The aneurysm treatment device of clause 16, wherein the first aneurysm filling portion is one of a plurality of aneurysm filling portions located on an outside of the stent portion.

Clause 18. The aneurysm treatment device of clause 17, wherein the plurality of aneurysm filling portions are arranged in one or more longitudinal patterns, one or more circumferential patterns, or both.

Clause 19. The aneurysm treatment device of clause 16, further comprising a plurality of radiopaque markers positioned on the stent portion; the plurality of radiopaque markers being positioned near a perimeter of the first aneurysm filling portion.

Clause 20. An aneurysm treatment device, comprising: a stent portion; and, a means for filling an aneurysm that is connected to the stent portion and located on an outside of the stent portion.

Clause 21. A method of delivering an aneurysm treatment device comprising: positioning an aneurysm treatment device near a mouth of an aneurysm; moving the aneurysm treatment device out of a delivery catheter so that a stent portion of the aneurysm treatment device radially expands and anchors within a blood vessel; and, expanding an aneurysm filling portion of the aneurysm treatment device into the aneurysm.

1. An aneurysm treatment device, comprising:
a stent portion; and,
a first aneurysm filling portion connected to the stent portion and located on an outside of the stent portion.

2. The aneurysm treatment device of claim 1, wherein the first aneurysm filling portion comprises one or more wires forming a three-dimensional spiral coil shape when in an expanded configuration.

3. The aneurysm treatment device of claim 2, wherein the three dimensional spiral coil comprises loops that progressively decrease in diameter away from the stent portion or that progressively increase in diameter away from the stent portion when in the expanded configuration.

4. The aneurysm treatment device of claim 3, further comprising a rounded top portion connected to an end region of the three dimensional spiral coil shape.

5. The aneurysm treatment device of claim 1, further comprising a rounded top portion connected to an end region of the first aneurysm filling portion; the rounded top portion comprising a metal, a polymer, or a hydrogel.

6. The aneurysm treatment device of claim 1, wherein the first aneurysm filling portion comprises a plurality of braided wires having a three-dimensional shape.

7. The aneurysm treatment device of claim 6, wherein the three-dimensional shape comprises a cylindrical shape, a rounded cylindrical shape, a spheroid shape, an oblate spheroid shape, a mound shape, a rounded dome shape, or a rounded pyramid shape.

8. The aneurysm treatment device of claim 1, further comprising a second aneurysm treatment portion; wherein the second aneurysm treatment portion is positioned on the outside of the stent portion radially opposite the first aneurysm filling portion.

9. The aneurysm treatment device of claim 1, further comprising a second aneurysm treatment portion; wherein the second aneurysm treatment portion is positioned on the outside of the stent portion and longitudinally adjacent the first aneurysm filling portion.

10. The aneurysm treatment device of claim 1, further comprising a second aneurysm treatment portion, a third aneurysm treatment portion, and a fourth aneurysm treatment portion positioned on the outside of the stent portion; wherein the first aneurysm filling portion, the second aneurysm treatment portion, the third aneurysm treatment portion, and the fourth aneurysm treatment portion are positioned along a circular pattern perpendicular to a longitudinal axis of the stent portion.

11. The aneurysm treatment device of claim 1, further comprising a plurality of radiopaque markers aligned at locations forming a pattern around a circumference of the stent portion, and near or between a distal edge of the aneurysm filling portion and/or near or between a proximal edge of the first aneurysm filling portion.

12. The aneurysm treatment device of claim 1, further comprising a plurality of radiopaque markers aligned at locations forming a pattern circumferentially around the first aneurysm filling portion.

13. The aneurysm treatment device of claim 1, wherein the first aneurysm filling portion comprises radiopaque DFT wires.

14. The aneurysm treatment device of claim 1, wherein the first aneurysm filling portion is attached to the stent portion by welding, adhesives, attachment coils, or by interweaving through each other.

15. The aneurysm treatment device of claim 1, wherein the first aneurysm filling portion has a compressed configuration and a radially expanded configuration that expands radially away from the stent portion.

16. An aneurysm treatment device, comprising:
a stent portion comprising one or more stent wires woven into a tubular shape; and,
a first aneurysm filling portion connected to the stent portion and located on an outside of the stent portion; wherein the first aneurysm filling portion has a compressed configuration and an expanded configuration forming a three-dimensional shape; and
wherein the three-dimensional shape comprises a cylindrical shape, a rounded cylindrical shape, a spheroid shape, an oblate spheroid shape, a mound shape, a rounded dome shape, or a rounded pyramid shape.

17. The aneurysm treatment device of claim 16, wherein the first aneurysm filling portion is one of a plurality of aneurysm filling portions located on an outside of the stent portion.

18. The aneurysm treatment device of claim 17, wherein the plurality of aneurysm filling portions are arranged in one or more longitudinal patterns, one or more circumferential patterns, or both.

19. The aneurysm treatment device of claim 16, further comprising a plurality of radiopaque markers positioned on the stent portion; the plurality of radiopaque markers being positioned near a perimeter of the first aneurysm filling portion.

20. An aneurysm treatment device, comprising:
a stent portion; and,
a means for filling an aneurysm that is connected to the stent portion and located on an outside of the stent portion.
